(19) United States
(12) Patent Application Publication
(10) Pub. No.: US 2016/0151251 A1
(43) Pub. Date: Jun. 2, 2016

(54) AQUEOUS OXIDIZING COMPOSITION

(71) Applicant: KAO GERMANY GMBH, Darmstadt (DE)

(72) Inventors: NIU JIAN, Darmstadt (DE); Dominic PRATT, Darmstadt (DE)

(73) Assignee: KAO GERMANY GMBH, Darmstadt (DE)

(21) Appl. No.: 14/898,606

(22) PCT Filed: Dec. 13, 2013
(86) PCT No.: PCT/EP2013/076481

§ 371 Date: Dec. 15, 2015

(30) Foreign Application Priority Data
Dec. 20, 2012 (EP) 12198688.9

(51) Int. Cl. A61K 8/06; A61K 8/34; A61K 8/58; A61K 8/31; A61K 8/92; A61K 8/46; A61Q 5/10; A61K 8/22

(52) U.S. Cl. CPC A61K 8/062; A61Q 5/10; A61K 8/342; A61K 8/22; A61K 8/31; A61K 8/922; A61K 8/463; A61K 8/58; A61K 2800/4324; A61K 2800/882

(57) ABSTRACT

Present invention relates to an aqueous oxidizing composition comprising at least one hydrophobic dye which provides improved mixing of such composition with an alkalizing composition prior to use and/or application such as onto hair.

Present invention relates to an aqueous oxidizing composition comprising at least one hydrophobic dye which provides improved mixing of such composition with an alkalizing composition prior to use and/or application such as onto hair.

Oxidizing compositions have been used in hair coloring and/or lightening for ages. They are mixed with an alkaline composition comprising at least one alkalizing agent which may additionally comprise one or more hair dye for either oxidative or direct dyeing hair. In case of a dyeing composition comprising oxidative dye precursors, the composition is practically colorless and mixing with another colorless composition may not be observed if the mixing is complete or not. The problem is aggravated when the two compositions mixed are viscous compositions because mixing with diffusion is practically negligible. On the other hand, any simple direct dye which colors the external phase of an oil in water emulsion composition may not simply be used because of possible interaction with hair and/or also reaction with the oxidizing agent and/or oxidative dye precursors. Therefore, there is a clear need to observe and control the mixing of the two aqueous compositions and especially two viscous aqueous compositions.

Furthermore, for achieving homogeneous dyeing of hair, coloring composition must be applied homogeneously onto hair. Since the compositions currently available are simply white or colorless, it is not possible to make sure that the composition is applied onto hair homogeneously. There is certainly need for a method which gives possibility to control that the dyeing composition is indeed applied onto the place to be colored and homogeneously.

Inventors of the present invention have found out unexpectedly that hydrophobic dyestuffs indeed make possible observing mixing efficiency of two compositions having elevated consistency and also have no interaction with hair and do not react with either oxidizing agent or with oxidative dye precursors. The same dyes also makes certain that the composition is applied at the parts to be colored homogeneously.

Accordingly, the first object of the present invention is an aqueous emulsion composition comprising one or more oxidizing agent, one or more fatty alcohol and/or oil, one or more surfactant and one or more dyestuff with a log P value at 25° C. higher than or equal to 2 wherein the composition is free of solid peroxides and/or persulphates and has a pH below 7.

The log P value used herein means that partition coefficient of the dyestuff between octanol and water at 25° C.

The second object of the present invention is the use of the composition of the present invention for lightening of hair color and/or for coloring hair.

The third object of the present invention is a process for lightening hair colour wherein the composition of the present invention is mixed with another composition comprising one or more alkalizing agent and applied onto hair and after leaving on hair for 5 to 45 min rinsed off from hair and optionally shampooed.

The fourth object of the present inventions is a process for colouring hair wherein the composition of the present invention is mixed with another composition comprising one or more alkalizing agent and one or more oxidative dye precursor applied onto hair and after leaving on hair for 5 to 45 min rinsed off from hair and optionally shampooed.

The fifth object of the present invention is a kit comprising two or more compositions wherein one of the compositions is the composition of the present invention.

Composition of the present invention is an aqueous emulsion composition and comprises one or more fatty alcohol and/or oil.

In principal any fatty alcohol is suitable for the purpose of the present invention. Suitable and non-limiting examples are according to the general structure

R₃₀—OH

wherein R₃₀ is a saturated or unsaturated, straight or branched alkyl chain having 12 to 30 C atoms which may also be substituted with one or more OH groups.

The non-limiting examples to suitable fatty alcohols are arachidyl alcohol, behenyl alcohol, brassica alcohol, C9-11 alcohols, C10-16 alcohols, C12-13 alcohols, C12-15 alcohols, C12-16 alcohols, C14-15 alcohols, C14-22 alcohols, C20-22 alcohols, caprylyl alcohol, cetearyl alcohol, cetyl alcohol, coconut alcohol, decyl alcohol, hydrogenated brassica alcohol, hydrogenated jojoba alcohol, hydrogenated rapeseed alcohol, hydrogenated tallow alcohol, hydroxystearyl alcohol, jojoba alcohol, lauryl alcohol, myristyl alcohol, oleyl alcohol, olive alcohol, palm alcohol, palm kernel alcohol, stearyl alcohol, tallow alcohol, and tridecyl alcohol and their mixtures.

Preferred are cetyl, stearyl, behenyl and cetearyl alcohols and their mixtures. The most preferred is cetearyl alcohol which is the mixture of cetyl and stearyl alcohols.

One or more fatty alcohol is used in the compositions of the present invention at a concentration 0.5 to 30%, preferably 1 to 25% more preferably 2 to 20%, most preferably 2.5 to 15% by weight calculated to the total composition.

The emulsion of the present invention comprises one or more oil either in addition to the one or more fatty alcohols or solely which are preferably liquid at room temperature (20° C.). One or more oil may be selected from synthetic and natural oils. Synthetic oils are silicones especially those of nonvolatile ones such as dimethicones with viscosity of 50 to 350 cSt measured by capillary viscosimeter and at 20° C., fatty acid fatty alcohol esters according to the general structure

R₁C(O)R₂

wherein R₁ is a straight or branched, saturated or unsaturated alkyl with 11 to 21 C atoms and R₂ is a straight or branched, saturated or unsaturated alkyl with 1 to 22 C atoms such as behenyl behenate, behenyl isostearte, butyl stearate, butyl oleate, butyl myristate, butyloctyl oleate, cetyl palmitate, cetyl myristate, cetyl oleate, cetyl caprylate, cetyl caprate, decyl oleate, decyl cocoate, decyl isostearate, ethylhexyl myristate, ethyl hexyl laurate, ethyl hexyl oleate, ethyl isostearte, ethyl laurate, ethyl linoleate, ethyl myristate, ethyl oleate, ethyl palmitate, ethylricinoleate, ethyl stearate, hexyl isostearet, hexyl laurate, hexyl myristate, hexyl stearate, hexyl decyl oleate, isobutyl laurate, isobutyl myristate, isobutyl palmitate, isobutyl stearate, isocetyl behenate, isobutyl laurate, isobutyl oleate, isobutyl stearate, isobutyl cocoate, isohexyl caprate, isopropyl palmitate, isopropyl stearate, isopropyl behenate, isopropyl laurate, isopropyl oleate, isopropyl ricinoleate and isopropyl palmitate, and fatty alcohol ethers according to general structure

R₃OR₄

wherein R₃ and R₄ are same or different, straight or branched, saturated or unsaturated alkyl with 8 to 22 C atoms such as dicetyl ether, dimyristyl ether, dicyprylyl ether and dodecyl ether.

Natural oils are mineral oil and plant derived oils such as avocado oil, olive oil, almond oil, peach oil, passiflora oil, black cumin oil, borage oils, evening primrose oil, grape seed oil, hempseed oil, kukui nut oil, rosehip oil, safflower oil, walnut oil and wheat germ oil. The most preferred is mineral oil.

Concentration of oil in the emulsions of the present invention is in the range of 1 to 20%, preferably 1 to 15% more preferably 1 to 10% and most preferably 1 to 5% by weight calculated to the total composition.

In the preferred embodiment of the present invention one or more fatty alcohol and one or more oil are used at a total concentration of 1 to 35%, preferably 1 to 30%, more preferably 1.5 to 25% and most preferably 2 to 20% by weight calculated to the total of the composition.

Further preferred embodiment of the present invention is that the weight ratio of the total fatty alcohol to total oil is less than or equal to 1.

The compositions of the present invention comprise one or more emulsifying surfactants also referred as emulgators. Suitable emulgators are anionic, cationic and nonionic ones and preferred are anionic and cationic surfactants and the most preferred are anionic surfactants.

Total concentration of the surfactants varies in the range of 0.05% to 15%, preferably 0.075 to 12.5%, more preferably 0.1 to 10 and most preferably 0.2% to 7.5% by weight, calculated to total composition.

Non-limiting suitable anionic surfactants are especially the known alkyl sulphates and alkyl ether sulfates, carboxylic acids, in particular in form of their alkali salts, as well as protein fatty acid condensates, fatty acid salts, alkyl/alkenyl succinates, anionic amino acid surfactants especially glutamates such as sodium lauroyl glutamate. The preferred anionic surfactants are alkyl sulphates and alkyl ether sulphates and the most preferred is alkyl sulfate types and their salts such as ammonium C12-15 alkyl sulphate, ammonium C12-16 alkyl sulphate, ammonium coco sulphate, ammonium lauryl sulphate, ammonium myristyl sulphate, magnesium coco sulphate, magnesium lauryl sulphate, magnesium coco/TEA sulphate, MEA lauryl sulphate, MIPA lauryl sulphate, potassium lauryl sulphate, sodium caprylyl sulphate, sodium cetearyl sulphate, sodium cetyl sulphate, sodium coco sulphate, sodium decyl sulphate, sodium ethylhexyl sulphate, sodium lauryl sulphate, sodium myristyl sulphate, sodium oleyl sulphate, sodium stearyl sulphate, sodium tridecyl sulphate, TEA coco sulphate, TEA lauryl sulphate, TEA oleyl sulphate and TIPA lauryl sulphate and their mixtures. Particularly preferred is sodium lauryl sulphate.

Suitable nonionic surfactants are in particular fatty alcohol polyglycol ethers according to general structure

R₁(OCH₂CH₂)ₙOH

R₁ is same as above and n is a number between 1 and 50, preferably 5 and 50, and more preferably 10 and 40 and most preferably 10 and 30.

Suitable non-limiting examples to non-ionic surfactants of fatty alcohol polyglycol ethers are ceteth-1, ceteth-2, ceteth-3, ceteth-4, ceteth-5, ceteth-6, ceteth-7, ceteth-10, ceteth-12, ceteth-13, ceteth-14, ceteth-15, ceteth-16, ceteth-17, ceteth-18, ceteth-18, ceteth-20, ceteth-23, ceteth-24, ceteth-25, ceteth-30, ceteth-40, ceteth-45, isoceteth-5, isoceteth-7, isoceteth-10, isoceteth-12, isoceteth-15, isoceteth-20, isoceteth-25, isoceteth-30, isosteareth-2, isosteareth-3, isosteareth-5, isosteareth-8, isosteareth-10, isosteareth-12, isosteareth-15, isosteareth-18, isosteareth-20, isosteareth-22, isosteareth-25, isosteareth-50, steareth-1, steareth-2, steareth-3, steareth-4, steareth-5, steareth-6, steareth-7, steareth-8, steareth-10, steareth-11, steareth-12, steareth-13, steareth-14, steareth-15, steareth-16, steareth-20, steareth-21, steareth-25, steareth-27, steareth-30, steareth-40, steareth-50, ceteareth-2, ceteareth-2, ceteareth-2, ceteareth-2, ceteareth-2, ceteareth-2, ceteareth-2, ceteareth-3, ceteareth-4, ceteareth-5, ceteareth-8, ceteareth-9, ceteareth-10, ceteareth-11, ceteareth-12, ceteareth-13, ceteareth-14, ceteareth-15, ceteareth-16, ceteareth-17, ceteareth-18, ceteareth-20, ceteareth-22, ceteareth-23, ceteareth-24, ceteareth-25, ceteareth-27, ceteareth-28, ceteareth-29, ceteareth-30, ceteareth-33, ceteareth-34, ceteareth-40, ceteareth-50, beheneth-2, beheneth-5, beheneth-10, beheneth-15, beheneth-20, beheneth-25, beheneth-30, oleth-2, oleth-3, oleth-4, oleth-5, oleth-6, oleth-7, oleth-8, oleth-9, oleth-10, oleth-11, oleth-12, oleth-15, oleth-16, oleth-20, oleth-23, oleth-24, oleth-25, oleth-30, oleth-35, oleth-40, oleth-44, oleth-45 and oleth-50 and their mixtures.

Further suitable non-ionic surfactants are fatty acid alkanolamides, amineoxides, and especially C₈-C₁₈-alkyl polyglucosides of the general structure

R₆—O—(R₇O)ₙO—Zₓ

wherein R₆ is an alkyl group with 8 to 18 carbon atoms, R₇ is an ethylene or propylene group, Z is a saccharide group with 5 to 6 carbon atoms, n is a number from 0 to 10 and x is a number between 1 and 5. Examples are decyl polyglucoside and cocoyl polyglucoside, both being commercially available.

Cationic surfactants especially mono alkyl quaternary ammonium salts of the following general structure

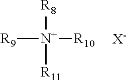

where R₈ is a saturated or unsaturated, branched or non-branched alkyl chain with 8-22 C atoms or

R₁₂CONH(CH₂)ₙ

where R₁₂ is saturated or unsaturated, branched or non-branched alkyl chain with 7-21 C atoms and n has typical value of 0-4 or

R₁₃COO(CH₂)ₙ

where R₁₃ is saturated or unsaturated, branched or non-branched alkyl chain with 7-21 C atoms and n has typical value of 0-4, and
and R₉, R₁₀ and R₁₁ are independent from each other H or lower alkyl chain with 1 to 4 carbon atoms, and X is an anion such as chloride, bromide or methosulfate.

Suitable non-limiting examples are cetrimonium chloride, steartrimonium chloride and behentrimonium chloride.

The compositions of the present invention comprise one or more oxidizing agent. Suitable and non-limiting examples are hydrogen peroxide, urea peroxide, melamine peroxide and sodium bromate and most preferably it is hydrogen peroxide preferably at a concentration of 1 to 20%, preferably 1 to 15% and more preferably 2 to 12% by weight calculated to the total of the composition, and preferably has a pH in the range of 2 to 5, more preferably 2 to 4.

Compositions of the present invention comprise one or more dyestuff with a log P value of more than equal to 2, preferably at a concentration in the range of 0.001 to 1%, more preferably 0.001 to 0.75% and most preferably 0.005 to 0.5% by weight calculated to the total of the composition.

Preferably the dyestuff with a log P value of more than or equal to 2 is free of transition metal ions such as copper and iron and free of anionic and/or cationic solubilizing groups such as —SO3-, —CO2- and —N+R20R21R22 wherein R20, R21 and R22 are same or different H, C1 to C4 alkyl, C2 to C4 alkyl which may be straight or branched, saturated or unsaturated which may additionally be substituted with one or more OH groups.

Suitable non-limiting examples are CI 73360, CI 61565, CI 60725, CI 26100, CI 12085 CI 47000, CI 11115, CI 26150, CI 11336, CI 61104, CI 61500 CI 46005, CI 12140, CI 61554 and CI 11680.

The emulsions of the present invention can comprises one or more polyol at a concentration in the range of 0.1 to 10%, preferably 0.5 to 7.5%, more preferably 0.75 to 5% and most preferably 1 to 5% by weight calculated to the total of the composition. The term polyol means any compound having 2 or more hydroxyl groups in its molecule. Suitable non-limiting examples are glycerin, 1,2-propylene glycol, polyglycerins with 2 to 10 glycerin units, panthenol, glycol, butyleneglycol, 1,2-butanediol, 1,4, butanediol, 2,3-butanediol, pentylene glycol and 1,5-pentanediol. Preferred are glycerin, 1,2-propylene glycol, glycol, butyleneglycol 1,2-butanediol, 1,4, butanediol, 2,3-butanediol, and panthenol. More preferred are glycerin, 1,2-propylene glycol, glycol, butyleneglycol, and panthenol. Most preferred are glycerin, 1,2-propylene glycol, and panthenol and particularly preferred polyol is glycerin.

For lightening hair color, the composition of the present invention is mixed with another composition comprising one or more alkalizing agent which is preferably also an emulsion and comprises one or more of the fatty alcohol, one or more of the oil and one or more of the surfactants as disclosed above at the above specified concentrations. Suitable are sodium or potassium hydroxide, ammonia, alkanol amines such as monoethanolamine, carbonates such as ammonium carbonate, potassium carbonate, ammonium bicarbonate and ammonium chloride. Concentration of one or more alkalizing agents in total is in the range of 1 to 10% by weight calculated to the total composition. The most preferred are ammonia and monoethanolamine. pH of the composition, preferably an emulsion composition, varies in the range of 6 to 10.5, preferably 6.5 to 10, more preferably 6.8 to 10 and most preferably 8 to 10.

In a further embodiment of the present invention the composition mixed with the composition of the present invention comprise one or more oxidative dye precursors. Suitable examples to the oxidative dye precursors are p-phenylenediamine, p-methylaminophenol and substituted p-phenylenediamines such as 2,5-diamino-toluene, 2-n-propyl or 2-ethyl-p-phenylenediamine, 2,6-di-methyl-p-phenylene-diamine, 2-(2,5-diaminophenyl) ethanol, 1-amino-4-bis-(2′-hydroxy-ethyl)amino-benzene, 2-(2-hydroxyethyl amino)-5-aminotoluene, 4,4′-diaminodiphenylamine, 4-aminodiphenylamine, 2-amino-5-N,N-diethyl aminotoluene, 4-amino-N-ethyl-N-isopropyl aniline, 2-chloro-p-phenylenediamine, 1-β-hydroxyethyl-2,5-diamino-4-chlorobenzene, 1-β-hydroxyethyl-2,5-diamino-4-methyl benzene, 2-methoxy-p-phenylenediamine, N,N-diethyl-p-phenylenediamine, 1-amino-4-β-methoxyethyl aminobenzene, 1-dimethyl-amino-4-aminobenzene, 1-hydroxy-2,5-diamino-4-methyl benzene, 1-hydroxymethyl-2,5-diaminobenzene, 1,3-dimethyl-2,5-diaminobenzene, 1,4-diamino isopropyl benzene and/or 1-amino-4-β-hydroxypropyl aminobenzene, pyrazole and the derivatives thereof such as 1-hydroxyethyl-4,5-diaminopyrazole, 3,4-diamino-5-hydroxypyrazole, 3,5-diaminopyrazole, 3,5-diamino pyrazol-1-carboxamide, 3-amino-5-hydroxypyrazole, 1-phenyl-2-methylpyrazole, 1-phenyl-3-methylpyrazole-5-one, 3,5-dimethylpyrazole, 3,5-dimethylpyrazole-1-methanol, 3,5-diamino-1,2,4-triazole, 4-aminophenol and the derivatives thereof such as 4-amino-3-methylphenol, 2-chloro-4-aminophenol, 2,6-dichloro-4-aminophenol, 2,4-diamino-phenol, 2,6-dibromo-4-aminophenol, tetraamino pyrimidines, triaminohydroxy pyrimidines, diaminomono- and -dihydroxy pyrimidines, aminotriazines, 5-amino salicylic acid and/or 1,2,4-triamino benzene or the water-soluble salts thereof.

Further suitable aminopyridines are 2,5-diaminopyridine, 2,3-diaminopyridine, 2,6-diaminopyridine, 3-amino-2-methyl amino-6-methoxypyridine, 2-dimethyl-5-aminopyridine, 2-dimethyl aminoethyl-3-hydroxypyridine, 2-amino-4,6-dimethyl pyridine, 2-amino-3-hydroxypyridine, 3-amino-2((3-hydroxyethyl amino)-6-methoxypyridine, 2,6-dimethyl amino-5-aminopyridine, 2-di(hydroxyethyl)amino-5-aminopyridine, 2-hydroxyethyl amino-5-aminopyridine, 4-hydroxy-2,5,6-triaminopyrimidine and/or the water-soluble salts thereof.

Further, indole and indoline derivatives can as well be contained in the colouring composition of the present invention. Examples to those are: 6-aminoindol, 6-hydroxyindole, 1-ethyl-6-hydroxyindole, 1-methyl-4-hydroxyindol, 1-methyl-6-hydroxyindole, 2-methyl-6-hydroxyindole, 5-hydroxyindol, 4-hydroxyindol, 5,6-dihydroxyindole, 6-aminoindoline, 6-hydroxyindoline, 1-ethyl-6-hydroxyindoline, 1-methyl-4-hydroxyindoline, 1-methyl-6-hydroxyindoline, 2-methyl-6-hydroxyindoline, 5-hydroxyindoline, 4-hydroxyindoline, 5,6-dihydroxyindoline and their respective salts.

Suitable coupling agents are resorcinol, 2-methyl resorcinol, 4-chlororesorcinol, 2-amino-4-chlorophenol, 5-amino-4-methoxy-2-methylphenol, 3-amino-phenol, 1-methyl-2-hydroxy-4-aminobenzene, 3-N,N-dimethyl aminophenol, 2.6-dihydroxy-3.5-dimethoxypyridine, 5-amino-3-methylphenol, 6-amino-3-methylphenol, 3-amino-2-methylamino-6-methoxypyridine, 2-amino-3-hydroxy-pyridine, 2-dimethyl-amino-5-aminopyridine, 2,6-diaminopyridine, 1,3-diamino-benzene, 1-amino-3-(2′-hydroxyethylamino)benzene, 1-amino-3-[bis(2′-hydroxy-ethyl)amino]benzene, α-naphthol, 4,6-dichlororesorcinol, 1,3-diamino-toluene, 1-hydroxy naphthalene, 4-hydroxy-1,2-methylenedioxy benzene, 1,5-dihydroxy naphthalene, 1,6-dihydroxy naphthalene, 1,7-dihydroxy naphthalene, 2,7-dihydroxy naphthalene, 1-hydroxy-2-methyl naphthalene, 4-hydroxy-1,2-methyldioxy benzene, 2,4-diamino-3-chlorophenol, 5-amino-2-methoxyphenol and/or 1-methoxy-2-amino-4-(2′-hydroxyethyl amino)benzene or the water-soluble salts thereof. 5-amino-2-methylphenol, 2-methyl-5-hydroxyethylaminophenol, 2,4-diamnophenoxyethanol, 2-amino-4-hydroxyethylaminoanisol, 2-methyl-5-amino-6-chlorophenol, 1,3-bis(2,4-diaminophenoxy)propane, 2-bis(2-hydroxyethyl)aminotoluene, 2-amino-5-methylphenol or the water-soluble salts thereof.

Concentration of one or more oxidative dyes in total—total concentration of precursors and couplers, if present—is in the range of 0.001 to 10% by weight, preferably 0.01 to 7.5% more preferably 0.05 to 5%, most preferably 0.1 to 3% by weight calculated to the total composition.

In a still further embodiment of the present invention, the composition mixed with the composition of the present invention comprises one or more direct dyes either alone or in combination with one or more oxidative dye precursors.

Suitable direct dyes are selected from cationic, anionic, neutral nitro dyes and their mixtures. Preferred are cationic and neutral nitro dyes and their mixtures.

Any cationic direct dye is in principal suitable for the compositions. Examples are Basic Blue 6, Basic Blue 7, Basic Blue 9, Basic Blue 26, Basic Blue 41, Basic Blue 99, Basic Brown 4, Basic Brown 16, Basic Brown 17, Natural Brown 7, Basic Green 1, Basic Orange 31, Basic Red 2, Basic Red 12 Basic Red 22, Basic Red 51, Basic Red 76, Basic Violet 1, Basic Violet 2, Basic Violet 3, Basic Violet 10, Basic Violet 14, Basic Yellow 57 and Basic Yellow 87.

Any anionic dye is in principal suitable for the compositions. Suitable examples are such as Acid Black 1, Acid Blue 1, Acid Blue 3, Food Blue 5, Acid Blue 7, Acid Blue 9, Acid Blue 74, Acid Orange 3, Acid Orange 6, Acid Orange 7, Acid Orange 10, Acid Red 1, Acid Red 14, Acid Red 18, Acid Red 27, Acid Red 50, Acid Red 52, Acid Red 73, Acid Red 87, Acid Red 88, Acid Red 92, Acid Red 155, Acid Red 180, Acid Violet 9, Acid Violet 43, Acid Violet 49, Acid Yellow 1, Acid Yellow 23, Acid Yellow 3, Food Yellow No. 8, D&C Brown No. 1, D&C Green No. 5, D&C Green No. 8, D&C Orange No. 4, D&C Orange No. 10, D&C Orange No. 11, D&C Red No. 21, D&C Red No. 27, D&C Red No. 33, D&C Violet 2, D&C Yellow No. 7, D&C Yellow No. 8, D&C Yellow No. 10, FD&C Red 2, FD&C Red 40, FD&C Red No. 4, FD&C Yellow No. 6, FD&C Blue 1, Food Black 1, Food Black 2, Disperse Black 9 and Disperse Violet 1 and their alkali metal salts such as sodium, potassium.

Among those, the preferred anionic dyestuffs are Acid Red 52, Acid Violet 2, Acid Red 33, Acid Orange 4, Acid Red 27 and Acid Yellow 10 and their salts. The most preferred anionic dyes are Acid Red 52, Acid Violet 2, Acid Red 33, Acid Orange 4 and Acid Yellow 10, and their salts

Neutral dyes, so called nitro dyes for shading purposes are also optionally contained in the compositions. Suitable ones are HC Blue No. 2, HC Blue No. 4, HC Blue No. 5, HC Blue No. 6, HC Blue No. 7, HC Blue No. 8, HC Blue No. 9, HC Blue No. 10, HC Blue No. 11, HC Blue No. 12, HC Blue No. 13, HC Brown No. 1, HC Brown No. 2, HC Green No. 1, HC Orange No. 1, HC Orange No. 2, HC Orange No. 3, HC Orange No. 5, HC Red BN, HC Red No. 1, HC Red No. 3, HC Red No. 7, HC Red No. 8, HC Red No. 9, HC Red No. 10, HC Red No. 11, HC Red No. 13, HC Red No. 54, HC Red No. 14, HC Violet BS, HC Violet No. 1, HC Violet No. 2, HC Yellow No. 2, HC Yellow No. 4, HC Yellow No. 5, HC Yellow No. 6, HC Yellow No. 7, HC Yellow No. 8, HC Yellow No. 9, HC Yellow No. 10, HC Yellow No. 11, HC Yellow No. 12, HC Yellow No. 13, HC Yellow No. 14, HC Yellow No. 15, 2-Amino-6-chloro-4-nitrophenol, picramic acid, 1,2-Diamino-4-nitrobenzol, 1,4-Diamino-2-nitrobenzol, 3-Nitro-4-aminophenol, 1-Hydroxy-2-amino-3-nitrobenzol and 2-hydroxyethylpicramic acid.

Concentration of one or more direct dyes in total is in the range of 0.001 to 10% by weight, preferably 0.01 to 7.5% more preferably 0.05 to 5%, most preferably 0.1 to 3% by weight calculated to total composition.

Any of the above mentioned compositions, composition of the present invention and also the composition mixed with the present invention, can comprise one or more of the substances disclosed below.

The composition may comprise ceramide type of compound such as cetyl-PG-hydroxyethylpalmitamide.

Further optional ingredient are sterols, especially the phytosterols are useful hair restructuring compounds can be present in the above mentioned compositions. Especially preferred ones are of plant origin for example ergosterol, sitosterol, stigmasterol, fucosterol, brassicasterol, fungisterol, campesterol, zymosterol, ascosterol, cerevisterol, episterol, faecosterol, spinasterol. Among those phytosterols, the ones found in “Avocadin” which is the unsaponified fraction of the avocado oil is more preferred.

In a further preferred embodiment of the present invention, compositions may comprises at least one diamine compound. Preferred diamide compounds are according to the general structure

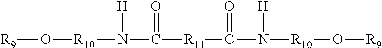

wherein R₉ is a linear or branched, saturated or unsaturated alkyl chain with 1 to 12 C atoms which may be substituted with hydroxy and/or alkoxy groups, preferably R₉ is linear or branched, saturated or unsaturated alkyl chain with 1 to 12 C atoms which may be substituted by 1 to 3 substituents selected from a hydroxy group and C1 to C6 alkoxy group, more preferably R₉ is a unsubstituted alkyl group with 1 to 12 C atoms, and alkyl group with 2 to 12 C atoms substituted by one or two hydroxyl groups, by one alkoxy group with 1 to 6 C atoms or by one hydroxyl and one alkoxy group with 2 to 6 C atoms, R₁₀ is linear or branched alkyl chain with 1 to 5 C atoms, preferably linear or branched alkyl chain with 2 to 5 C atoms and more preferably an alkyl chain with 2 to 3 C atoms, and R₁₁ linear or branched, saturated or unsaturated alkyl chain with 1 to 22 C atoms, preferably linear or branched, saturated or unsaturated alkyl chain with 11 to 22 C atoms.

Preferred individual diamide compounds are the ones according to the formula A to G.

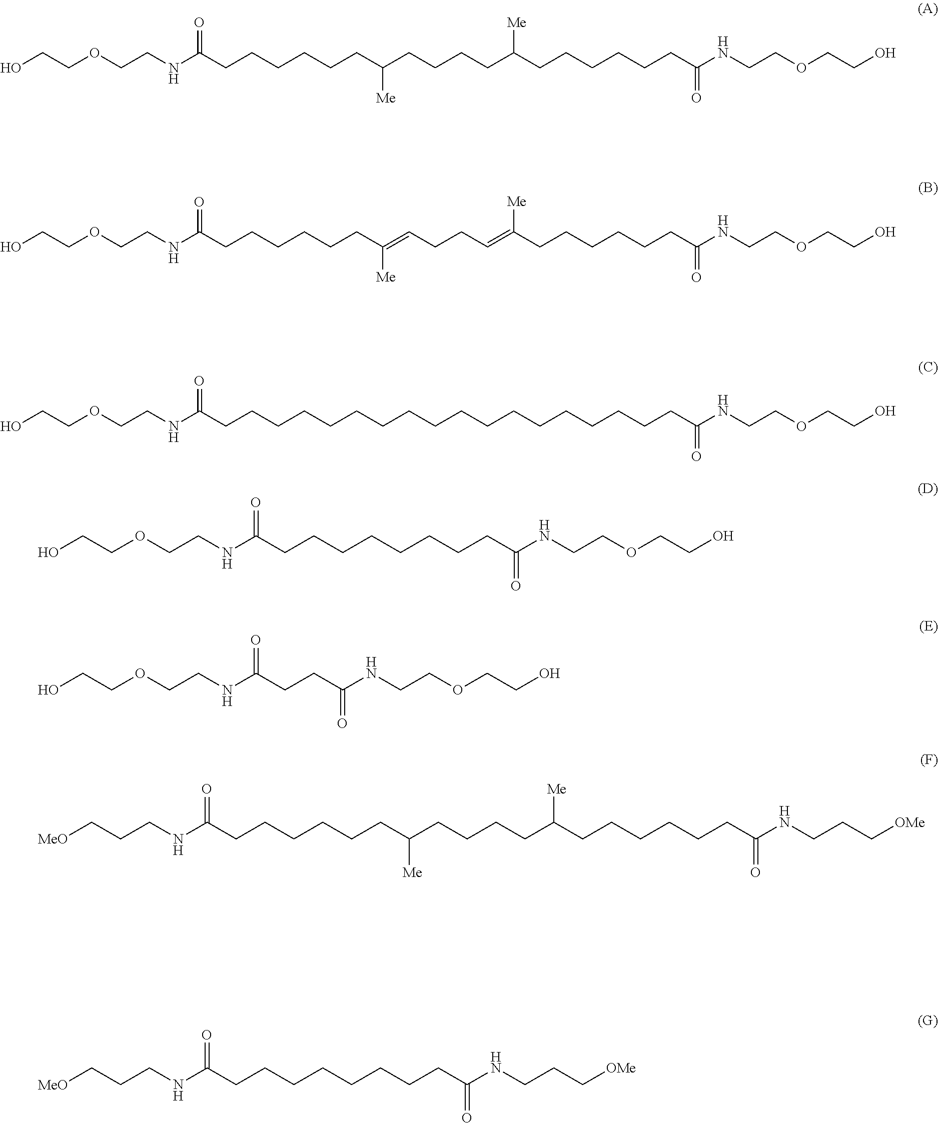

Particularly preferred diamide compound is the compound F which is bis (methoxypropylamido)isodocosane and commercially available from Kao Corporation—Japan.

Concentration of diamide compounds in the intermediate treatment compositions of the present invention is in the range of 0.001 to 5%, preferably 0.002 to 3% more preferably 0.005 to 2% and most preferably 0.01 to 1% by weight calculated to the total composition.

Further additional compounds may be present in the above mentioned compositions of the present invention is ubichinone of the formula

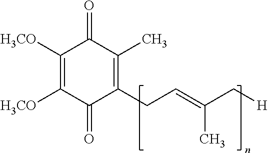

where n is a number between 1 and 10. Preferred ubichinones are the ones where n is a number between 6 and 10 and especially preferred is Ubichinone 50 where n is 10, also known as Coenzyme Q10. Concentration ubichinone of the above formula in the compositions is from 0.0001 to 1%, preferably from 0.0002 to 0.75%, more preferably from 0.0002 to 0.5% and most preferably from 0.0005 to 0.5% by weight, calculated to the total composition.

The composition of the present invention may further comprise the compounds found customarily in such compositions such as chelating agents such as EDTA and/or etidronic acid and/or their salts, pH adjusting agents such as organic and/or inorganic acids and/or their salts, preservatives, oxidizing agent stabilizing agents such as acetaminophen and/or salicylic acid, fragrance and additional compounds especially improving hair conditions.

In a further embodiment of the present invention the composition has a viscosity in the range of 500 to 50000 mPa·s, preferably 750 to 40000 mPa·s, more preferably 1000 to 35000 mPa·s, and most preferably 2000 to 30000 mPa·s, and in particular 5000 to 20000 mPa·s measured at 20° C. with a Brookfield viscosimeter with a suitable Spindle such as Spindle 5.

In a still further preferred embodiment of the present invention the composition mixed with the composition of the present invention has the viscosity in the range of 500 to 50000 mPa·s, preferably 750 to 40000 mPa·s, more preferably 1000 to 35000 mPa·s, and most preferably 2000 to 30000 mPa·s, and in particular 5000 to 20000 mPa·s measured at 20° C. with a Brookfield viscosimeter with a suitable Spindle such as Spindle 5.

The following examples are to illustrate but not to limit the invention.

### EXAMPLE 1

### Oxidizing Emulsion Composition

| | % by weight |
|---|---|
| Cetearyl alcohol | 10.0 |
| Mineral oil | 3.5 |
| Sodium lauryl sulphate | 2.0 |
| Hydrogen peroxide | 9.0 |
| CI 73360 | 0.01 |
| Salicylic acid | 0.05 |
| Etidronic acid | 0.3 |
| Phosphoric acid/sodium phosphate | q.s. to pH 2.5 |
| Water | to 100 |

The above composition had a viscosity of approximately 15000 mPa·s measured with Brookfield viscosimeter Spinle 4 at 20° C.

The above composition was mixed with a commercially available composition under the trade name Topchic which comprises alkalizing agent and one or more oxidative dye precursors and it is an emulsion. It was observed that the red dye CI 73360 made possible to observe the completeness of the mixing and also application of thus obtained composition delivered homogeneous colour on hair. It was not possible to observe homogeneity of the composition prior to application onto hair without the red dye. Additionally it was observed that addition of red dye into the composition did not result in change of hair colour.

Similar observations were made with the examples below.

### EXAMPLE 2

### Oxidizing Emulsion Composition

| | % by weight |
|---|---|
| Cetearyl alcohol | 10.0 |
| Olive oil | 3.5 |
| Ceteareth-20 | 4.0 |
| Glycerine | 2.0 |
| Hydrogen peroxide | 9.0 |
| CI 60725 | 0.03 |
| Salicylic acid | 0.05 |
| Etidronic acid | 0.3 |
| Phosphoric acid/sodium phosphate | q.s. to pH 2.5 |
| Water | to 100 |

### EXAMPLE 3

### Oxidizing Emulsion Composition

| | % by weight |
|---|---|
| Cetearyl alcohol | 10.0 |
| Olive oil | 3.5 |
| Ceteareth-20 | 4.0 |
| Glycerine | 2.0 |
| Hydrogen peroxide | 9.0 |
| CI 47000 | 0.1 |
| Salicylic acid | 0.05 |
| Etidronic acid | 0.3 |
| Phosphoric acid/sodium phosphate | q.s. to pH 2.5 |
| Water | to 100 |

1. An aqueous emulsion composition comprising:
one or more oxidizing agent,
one or both of: one or more fatty alcohol and one or more oil,
one or more surfactant, and
one or more dyestuff having a log P value at 25° C. of higher than or equal to 2,
wherein the composition is free of solid peroxides or persulphates, or free of both solid peroxides and persulphates, and has a pH below 7.

2. The composition according to claim 1, wherein the one or more oxidizing agent is hydrogen peroxide and present at a concentration of 1 to 20% by weight calculated to the total composition.

3. The composition according to claim 1, wherein the one or more fatty alcohol is selected from compounds according to general structure
R₃₀—OH
wherein R₃₀ is a saturated or unsaturated, straight or branched alkyl chain having 12 to 30 C atoms which may also be substituted with one or more OH groups.

4. The composition according to claim 3, wherein the one or more fatty alcohol is selected from arachidyl alcohol, behenyl alcohol, brassica alcohol, C9-11 alcohols, C10-16 alcohols, C12-13 alcohols, C12-15 alcohols, C12-16 alcohols, C14-15 alcohols, C14-22 alcohols, C20-22 alcohols, caprylyl alcohol, cetearyl alcohol, cetyl alcohol, coconut alcohol, decyl alcohol, hydrogenated brassica alcohol, hydrogenated jojoba alcohol, hydrogenated rapeseed alcohol, hydrogenated tallow alcohol, hydroxystearyl alcohol, jojoba alcohol, lauryl alcohol, myristyl alcohol, oleyl alcohol, olive alcohol, palm alcohol, palm kernel alcohol, stearyl alcohol, tallow alcohol, tridecyl alcohol and mixtures thereof.

5. The composition according to claim 1, wherein the one or more fatty alcohol is present at a concentration of 0.5 to 30%, by weight calculated to the total composition.

6. The composition according to claim 1, wherein the one or more oil is selected from synthetic oils and natural oils, and
wherein synthetic oils are selected from silicones, fatty acid fatty alcohol esters according to the general structure
R₁C(O)R₂
wherein R₁ is a straight or branched, saturated or unsaturated alkyl with 11 to 21 C atoms and R₂ is a straight or branched, saturated or unsaturated alkyl with 1 to 22 C atoms, and fatty alcohol ethers according to general structure
R₃OR₄
wherein R₃ and R₄ are same or different, straight or branched, saturated or unsaturated alkyl with 8 to 22 C atoms,
and natural oils are selected from mineral oils and plant derived oils.

7. The composition according to claim 1, wherein the one or more oil is present at a concentration in the range of 1 to 20% by weight calculated to the total composition.

8. The composition according to claim 1, wherein the one or more fatty alcohol and the one or more oil are present at a total concentration in the range of 1 to 35% by weight calculated to the total composition.

9. The composition according to claim 1, wherein the one or more surfactant selected from anionic, cationic and nonionic surfactants,
wherein
the anionic surfactants are selected from alkyl sulphates, alkyl ether sulfates, carboxylic acids or alkali salts thereof, fatty acid salts, alkyl/alkenyl succinates, and anionic amino acid surfactants, and
the nonionic surfactants are selected from fatty alcohol polyglycol ethers according to general structure
R₃₀(OCH₂CH₂)ₙOH
wherein R₃₀ is same as above and n is a number between 1 and 50, fatty acid alkanolamides, amineoxides, and C₈-C₁₈-alkyl polyglucosides of the general structure
R₆—O—(R₇O)ₙO—Zₓ
wherein R₆ is an alkyl group with 8 to 18 carbon atoms, R₇ is an ethylene or propylene group, Z is a saccharide group with 5 to 6 carbon atoms, n is a number from 0 to 10 and x is a number between 1 and 5, and
the cationic surfactants are mono alkyl quaternary ammonium salts of the following general structure 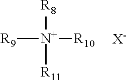
where R₈ is a saturated or unsaturated, branched or non-branched alkyl chain with 8-22 C atoms, or
R₁₂CONH(CH₂)ₙ
where R₁₂ is saturated or unsaturated, branched or non-branched alkyl chain with 7-21 C atoms and n has typical value of 0-4, or
R₁₃COO(CH₂)ₙ
where R₁₃ is saturated or unsaturated, branched or non-branched alkyl chain with 7-21 C atoms and n has typical value of 0-4,
and R₉, R₁₀ and R₁₁ are independent from each other H or lower alkyl chain with 1 to 4 carbon atoms, and X is an anion.

10. The composition according to claim 1, wherein the one or more dyestuff is free of transition metal ions, and free of anionic or cationic solubilizing groups or both anionic and cationic solubilizing groups.

11. (canceled)

12. A process for lightening hair color comprising:
(a) mixing a first composition according to claim 1 with second composition comprising one or more alkalizing agent,
(b) applying a mixture obtained from step a) onto hair,
(c) leaving the mixture obtained from step a) on hair for 5 to 45 min, and
(d) rinsing the mixture obtained from step a) off from hair.

13. The process according to claim 12, wherein the second composition further comprises one or more oxidative dye precursor.

14. The process according to claim 12, wherein the first composition has a viscosity in the range of 500 to 50000 mPa·s, and the second composition has a viscosity in the range of 500 to 50000 mPa·s, measured at 20° C. with a Brookfield viscosimeter with a suitable spindle.

15. A kit comprising two or more compositions, wherein one of the two or more compositions is a composition according to claim 1.

16. The composition according to claim 4, wherein the one or more fatty alcohol is a mixture of cetyl and stearyl alcohols.

17. The composition of claim 5, wherein the one or more fatty alcohol is present at a concentration of 1 to 25% by weight calculated to the total composition.

18. The composition of claim 6, wherein the one or more oil is a nonvolatile silicone.

19. The composition of claim 7, wherein the one or more oil is present at a concentration in the range of 1 to 15% by weight calculated to the total composition.

20. The composition of claim 8, wherein the one or more fatty alcohol and the one or more oil are present at a total concentration in the range of 1 to 30% calculated to the total composition.

21. The composition of claim 9, wherein the one or more surfactant is present at a total concentration in the range of 0.05% to 15% by weight calculated to total composition.
